(19) United States
(12) Patent Application Publication
(10) Pub. No.: US 2020/0170918 A1
(43) Pub. Date: Jun. 4, 2020

(54) NATURALLY THICKENED COSMETIC CLEANING AGENTS

(71) Applicant: Henkel AG & Co. KGaA, Duesseldorf (DE)

(72) Inventors: SAMEH HASAN FARES, Hilden (DE); HEIKE SCHELGES, Willich (DE)

(73) Assignee: Henkel AG & Co. KGaA, Duesseldorf (DE)

(21) Appl. No.: 16/667,238

(22) Filed: Oct. 29, 2019

(30) Foreign Application Priority Data
Dec. 3, 2018 (DE) 10 2018 220 854.9

(51) Int. Cl. A61K 8/73; A61Q 5/02; A61Q 19/00

(52) U.S. Cl. CPC A61K 8/732; A61K 8/737; A61Q 5/02; A61K 2800/596; A61K 2800/30; A61K 2800/48; A61Q 19/00

(57) ABSTRACT

The present disclosure relates to cosmetic cleaning agents which, based in each case on their weight, contain from about 1 to about 30% by weight of surfactant(s); from about 0.1 to about 5% by weight of guaran (INCI name Guar Gum); from about 0.1 to about 5% by weight of xanthan (INCI name Xanthan Gum); from 0 to about 10% by weight of unmodified starch, from 0 to about 10% by weight of modified starch(es), with the proviso that the total amount of the two latter ingredients is from about 0.1 to about 10% by weight.

### CROSS-REFERENCE TO RELATED APPLICATION

This application claims priority to German Patent Application No. 10 2018 220 854.9, filed Dec. 3, 2018, which is incorporated herein by reference in its entirety.

### TECHNICAL FIELD

The present disclosure relates to surfactant cleaning agents for the skin and hair, which are viscosity-enhanced with a thickener system based on renewable raw materials.

### BACKGROUND

Increased viscosity is an advantage when applying cosmetic cleaning agents for skin and hair, these products can thus be used sparingly and be distributed well. In addition, the consumer associates increased viscosity products with an increased performance in terms of cleaning and care. Special gel-forming thickeners are also required to stabilize solid particles such as pigments or peels and nourishing oils and waxes in the cleaning agent.

Although thickening agents based on synthetic polymers used in cosmetics are readily biodegradable and do not enter the surface waters as solids, they are increasingly critically considered by larger consumer groups and in some cases mistakenly associated with the microplastic problem. Paradoxically, the consumer is not ready to accept changes in his habits of use, but desires products that fully correspond to his, although partly false, ecological notions, but at the same time fully match to the products known to him in their haptic and other properties.

“Natural” thickening agents, that is, thickeners based on renewable raw materials have long been known in the field of cosmetics and are widely used. Examples here include agar, carrageenan, amylopectin, xanthan gum, guar gum, starch, cellulose, alginates, etc. Not only are the unchanged “natural” substances used here, but also chemically modified products such as hydroxypropyl starch, methylcellulose, hydroxyethylcellulose, hydroxypropyl guar, etc. In some cases, further functions are integrated into the molecule by chemical modification in addition to the viscosity regulation, such as, for example, with guar hydroxypropyltrimonium chloride.

Thickeners based on renewable raw materials are often inferior to synthetic polymers in their performance, whether in the thickening effect, the long-term stability of the corresponding cosmetic formulations or the haptics when applying the product. Products containing natural thickeners are often perceived as “sticky” and do not exhibit the desired lotion-like flow behavior.

There is therefore a need to use thickeners based on renewable raw materials in cosmetic cleaning agents for the skin and hair instead of the commonly used synthetic polymers, which give the consumer the usual image in terms of thickening performance, stability of the products and application properties.

### BRIEF SUMMARY

Cosmetic cleaning agents are provided herein. In an exemplarily embodiment, a cosmetic cleaning agent includes, based on a total weight of the cosmetic cleaning agent, from about 1 to about 30% by weight of surfactant, from about 0.1 to about 5% by weight of guaran, from about 0.1 to about 5% by weight of xanthan, from 0 to about 10% by weight of unmodified starch, and from 0 to about 10% by weight of modified starch. A total amount of ingredients d) and e) is from about 0.1 to about 10% by weight.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure has the object of solving the above-mentioned problems. The problem underlying the present disclosure is solved by the subject matter of claim 1. A first subject of the present disclosure is therefore a cosmetic cleaning agent containing—in each case based on its weight—

a) from about 1 to about 30% by weight of surfactant(s);
b) from about 0.1 to about 5% by weight of guaran (INCI name Guar Gum);
c) from about 0.1 to about 5% by weight of xanthan (INCI name Xanthan Gum);
d) from 0 to about 10% by weight of unmodified starch
e) from 0 to about 10% by weight of modified starch(es),
with the proviso that the total amount of ingredients d) and e) is from about 0.1 to about 10% by weight.

The cleaning agents as contemplated herein contain the ingredients in a suitable carrier. A suitable carrier preferably is understood as meaning an aqueous or aqueous-alcoholic carrier. Preferably, the carrier contains at least about 50% by weight, more preferably at least about 60% by weight, and most preferably at least about 70% by weight of water. Furthermore, the cosmetic carrier can contain from about 0.01 to about 40% by weight, preferably from about 0.05 to about 35% by weight and in particular from about 0.1 to about 30% by weight of at least one alcohol which can be selected from ethanol, 1-propanol, 2-propanol, isopropanol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol or mixtures of these alcohols. Preference is given to the water-soluble alcohols. Particularly preferred are ethanol, 1-propanol, 2-propanol, isopropanol, 1,2-propylene glycol, glycerol, benzyl alcohol and/or phenoxyethanol and mixtures of these alcohols. Especially preferred is glycerol.

The cleaning agents as contemplated herein contain from about 1 to about 30% by weight surfactant(s) as a first ingredient. They therefore contain at least one surfactant in the amount mentioned above or two surfactants or a plurality of surfactants in a total amount corresponding to the amount mentioned above. Preferably, the surfactant(s) are used in narrower ranges of amounts. Here, preferred cleaning agents as contemplated herein contain from about 1.5 to about 25% by weight, preferably from about 2 to about 20% by weight, more preferably from about 2.5 to about 15% by weight, even more preferably from about 3 to about 12.5% by weight and in particular from about 3.5 to about 10% by weight of surfactant(s).

In principle, the cleaning agents as contemplated herein can contain all known surfactants. Typical examples of anionic surfactants are soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and their salts, fatty acid isethionates, fatty acid sarcosinates, fatty acid taurides, N-acyl amino acids such as acyl lactylates, acyl tartrates, acyl glutamates and acyl aspartates, alkyl oligoglucoside sulfates, protein fatty acid condensates (in particular vegetable products based on wheat) and alkyl (ether) phosphates. If the anionic surfactants contain polyglycol ether chains, these can have a conventional, but preferably a narrow homolog distribution. Typical examples of nonionic surfactants are fatty alcohol polyglycol ethers, alkylphenol polyglycol ethers, fatty acid polyglycol ethers, fatty acid amide polyglycol ethers, fatty amine polyglycol ethers, alkoxylated triglycerides, mixed ethers or mixed formals, optionally partially oxidized alk(en)yloligoglycosides or glucuronic acid derivatives, fatty acid N-alkylglucamides, protein hydrolysates (in particular vegetable products based on wheat), polyol fatty acid esters, sugar esters, sorbitan esters, polysorbates and amine oxides. If the nonionic surfactants contain polyglycol ether chains, these can have a conventional, but preferably a narrow homolog distribution. Typical examples of cationic surfactants are quaternary ammonium compounds and ester quats, in particular quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines.

In view of the problem mentioned at the outset and the skin-friendliness of the agents, it has proved to be advantageous when only surfactants from certain groups of substances are used. Cosmetic cleaning agents as contemplated herein that have been proven to be particularly advantageous in terms of consumer acceptance, skin-friendliness and application properties contain exclusively surfactant(s) from the groups of

alkyl oligo- and polysaccharides

sulfosuccinates

isethionates

taurides

acyl glutamates

glycinates

betaines.

Alkyl and alkenyl oligoglycosides are known nonionic surfactants, which can be described by the formula

R¹O[G]ₚ

in which R¹ stands for an alkyl and/or alkenyl radical having 4 to 22 carbon atoms, G stands for a sugar radical having 5 or 6 carbon atoms and p stands for a number from about 1 to about 10. They can be obtained according to the relevant methods of preparative organic chemistry.

The alkyl and/or alkenyl oligoglycosides can be derived from aldoses or ketoses having 5 or 6 carbon atoms, preferably glucose. The preferred alkyl and/or alkenyloligo glycosides are thus alkyl and/or alkenyloligo glucosides. The index number p in the general formula indicates the degree of oligomerization (DP), that is, the distribution of mono- and oligoglycosides, and stands for a number between from about 1 and about 10. While p always has to be an integer in a given compound, and here can primarily assume the values p=from 1 to 6, the value p for a given alkyl oligoglycoside is an analytically determined arithmetic quantity, which usually represents a fractional number. Preference is given to using alkyl and/or alkenyl oligoglucosides having a mean degree of oligomerization p of from about 1.1 to about 3.0. From an application point of view, those alkyl and/or alkenyl oligoglucosides whose degree of oligomerization is less than about 1.7 and in particular between about 1.2 and about 1.4 are preferred. The alkyl or alkenyl radical R¹ can be derived from primary alcohols having 4 to 11, preferably 8 to 10 carbon atoms. Typical examples are butanol, caproic alcohol, caprylic alcohol, capric alcohol and undecyl alcohol and technical mixtures thereof, as obtained, for example, in the hydrogenation of technical fatty acid methyl esters or in the hydrogenation of aldehydes from Roelen's oxo synthesis. Preference is given to alkyloligo glucosides of chain length C₈-C₁₀ (DP=1 to 3), which are obtained as a forerunnings in the distillative separation of technical C₈-C₁₈ coco fatty alcohol and can have impurities with a proportion of less than about 6% by weight C₁₂ alcohol and alkyl oligoglucosides based on technical C_{9/11} oxo alcohols (DP=1 to 3). The alkyl or alkenyl radical R¹ can be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which can be obtained as described above. Preference is given to alkyl oligoglucosides based on hydrogenated C_{12/14} coconut alcohol having a DP of from about 1 to about 3.

Preferred cleaning agents as contemplated herein contain from about 0.1 to about 10% by weight, preferably from about 0.25 to about 7.5% by weight, more preferably from about 0.5 to about 5% by weight, even more preferably from about 0.75 to about 2.5% by weight and in particular from about 1 to about 2% by weight surfactant(s) from the group of alkyl oligo- and polysaccharides.

Sulfosuccinates, also referred to as sulfosuccinic acid esters, can be described by the formula

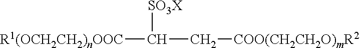

in which R¹ stands for an alkyl and/or alkenyl radical having 6 to 22 carbon atoms, R² stands for R¹ or X, m and n are each independently 0 or numbers from 1 to 10 and X stands for an alkali or alkaline earth metal, ammonium, alkylammonium, alkanolammonium or glucammonium. Typical examples are sulfosuccinic acid mono- and/or diesters in the form of their sodium salts, which are derived from fatty alcohols having 8 to 18, preferably 8 to 10 or 12 to 14 carbon atoms; the fatty alcohols can be etherified with on average from about 1 to about 10 and preferably from about 1 to about 5 moles of ethylene oxide and have both a conventional and preferably a narrowed homolog distribution. Examples would include di-n-octylsulfosuccinat and monolauryl-3EO-sulfosuccinate in the form of their sodium salts.

Preferred cleaning agents as contemplated herein contain from about 0.5 to about 15% by weight, preferably from about 1 to about 10% by weight, more preferably from about 1.5 to about 7.5% by weight, even more preferably from about 2 to about 6% by weight and in particular from about 2.5 to about 5% by weight of surfactant(s) from the group of sulfosuccinates.

Isethionates can be described by the formula (I)

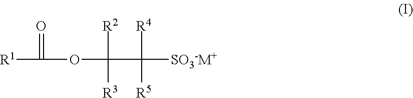

in which the radicals R² to R⁵ in each case stand for a hydrogen atom, or in which at least one of the radicals R² to R⁵ stands for a C₁-C₄ alkyl radical, and the other radicals independently of one another stand for a hydrogen atom or a C₁-C₄ alkyl radical, wherein—R¹ in each case stands for a linear or branched, saturated or unsaturated alkyl radical having 6 to 30 carbon atoms, and—M+ in each case stands for an ammonium, an alkanalammonium or a metal cation.

Preferred anionic surfactants of the abovementioned formula (I) have a linear or branched, saturated or unsaturated alkyl radical having 8 to 18 carbon atoms as radical R¹. Particularly preferably, the radical R¹ stands for a C₈, C₁₀, C₁₂, C₁₄, C₁₆ radical or mixtures of these fatty acid radicals as obtained when the fatty acid(s) is/are derived from natural oils such as coconut oil(s). In preferred anionic surfactants according to the formula (I), M+ preferably stands for an alkali metal cation or an ammonium ion, particularly preferably a potassium or a sodium ion and particularly preferably a sodium ion.

Very particularly preferred anionic surfactants according to the abovementioned formula (I) are those compounds known under the INCI names Sodium Cocoyl Isethionate, Potassium Cocoyl Isethionate, Ammonium Cocoyl Isethionate, Sodium Lauroyl Isethionate, Potassium Lauroyl Isethionate, Ammonium Lauroyl Isethionate, Sodium Myristoyl Isethionate, Potassium Methyl Isethionate and Ammonium Myristoyl Isethionate. Particularly preferred are Sodium Cocoyl Isethionate and/or Sodium Lauroyl Isethionate. Corresponding commercial products are available, for example, from the companies Clariant or Uniquema under the trade names “Hostapon®” or “Arlatone®”.

Further preferred anionic surfactants of the abovementioned formula (I) have a linear or branched, saturated or unsaturated alkyl radical having 8 to 18 carbon atoms as radical R¹. Particularly preferably, the radical R¹ stands for a C₈, C₁₀, C₁₂, C₁₄, C₁₆ radical or mixtures of these fatty acid radicals as obtained when the fatty acid(s) is derived from natural oils such as coconut oil(s). M+ in preferred anionic surfactants of the formula (I) preferably stands for an alkali metal cation or an ammonium ion, particularly preferably for a potassium or a sodium ion and particularly preferably for a sodium ion, wherein at the same time the radicals R² to R⁵ each stand for a methyl, ethyl, n-propyl, n-butyl or 2-butyl group and at least one of the radicals R² to R⁵ stands for a methyl, ethyl or n-propyl group and in particular a methyl group. In a particularly preferred embodiment, only one of the radicals R² to R⁵ stands for a C₁-C₄ alkyl group, in particular a methyl group, and the other radicals each stand for a hydrogen atom. In principle, it is also possible that the anionic surfactant of the formula (I) contains an isomer mixture in which both components are present, which, for example, have as the radical R² a C₁-C₄ alkyl group, in particular a methyl group, and as radicals R³ to R⁵ each a hydrogen atom, as well as components which, for example, have as the radical R⁵ a C₁-C₄ alkyl group, in particular a methyl group, and as radicals R² to R⁴ each a hydrogen atom.

Very particularly preferred anionic surfactants of the aforementioned formula (I) are those known under the INCI names Sodium Cocoyl Methyl Isethionate, Potassium Cocoyl Methyl Isethionate, Ammonium Cocoyl Methyl Isethionate, Sodium Lauroyl Methyl Isethionate, Potassium Lauroyl Methyl Isethionate, Ammonium Lauroyl Methyl Isethionate, Sodium Myristoyl Methyl Isethionate, Potassium Myristoyl Methyl Isethionate and Ammonium Myristoyl Methyl Isethionate. Particular preference is given to Sodium Cocoyl Methyl Isethionate and/or Sodium Lauroyl Methyl Isethionate. Corresponding commercial products are available, for example, from lnnospec under the trade name “Lselux® LQ-CLR-SB”.

Preferred cleaning agents as contemplated herein contain from about 0.5 to about 15% by weight, preferably from about 1 to about 10% by weight, more preferably from about 1.5 to about 7.5% by weight, even more preferably from about 2 to about 6% by weight and in particular from about 2.5 to about 5% by weight of surfactant(s) from the group of isethionates.

Taurides (also N-acyl taurides, N-methyl-N-acyl taurates, N-acyl taurates, or N-acyl taurines) are a group of mild anionic surfactants. Their hydrophilic head group includes N-methyltaurine (2-methylaminoethanesulfonic acid), the lipophilic radical of a carboxylic acid linked via an amide bond long-chain (fatty acid). The fatty acids used are lauric (C₁₂), myristic (C₁₄), palmitic (C₁₆), and stearic acid (C₁₈), but mainly oleic acid (C_{18:1}) and coconut fatty acid mixture (C₈-C₁₈),

The general structural formula for taurides is:

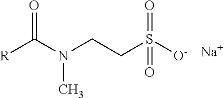

R is an odd-numbered alkyl radical CₙH₂ₙ₊₁ with n=7-17. In addition to sodium as a cation, counterions such as ammonium or other alkali or alkaline earth metals play no special role.

Preferred cleaning agents as contemplated herein contain from about 0.5 to about 15% by weight, preferably from about 1 to about 10% by weight, more preferably from about 1.5 to about 7.5% by weight, even more preferably from about 2 to about 6% by weight and in particular from about 2.5 to about 5% by weight of surfactant(s) from the group of taurides.

Betaines are known surfactants, which are predominantly produced by carboxyalkylation, preferably carboxymethylation of aminic compounds. Preferably, the starting materials are condensed with halocarboxylic acids or their salts, in particular with sodium chloroacetate, one mole of salt being formed per mole of betaine. Furthermore, the addition of unsaturated carboxylic acids, such as acrylic acid, is possible. Examples of suitable betaines are the carboxyalkylation products of secondary and in particular tertiary amines which can be described by the formula

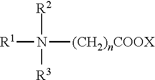

in which R¹ stands for alkyl and/or alkenyl radicals having 6 to 22 carbon atoms, R² stands for hydrogen or alkyl radicals having 1 to 4 carbon atoms, R³ stands for alkyl radicals having 1 to 4 carbon atoms, n stands for a number from 1 to 6 and X stands for an alkali and/or alkaline earth metal or ammonium. Typical examples are the carboxymethylation products of hexylmethylamine, hexyldimethylamine, octyldimethylamine, decyldimethylamine, dodecylmethylamine, dodecyldimethylamine, dodecylethylmethylamine, C_{12/14} cocoalkyldimethylamine, myristyldimethylamine, cetyldimethylamine, stearyldimethylamine, stearylethylmethylamine, oleyldimethylamine, C_{16/18} tallowalkyldimethylamine, and technical mixtures thereof.

Also considered are carboxyalkylation products of amidoamines, which can be described by the formula

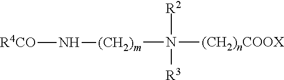

in which R⁴CO stands for an aliphatic acyl radical having 6 to 22 carbon atoms and 0 or 1 to 3 double bonds, m stands for a number from 1 to 3 and R², R³, n and X have the meanings given above. Typical examples are reaction products of fatty acids having 6 to 22 carbon atoms, namely caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid and their technical mixtures, with N,N-dimethylaminoethylamine, N,N-dimethylaminopropylamine, N,N-diethylaminoethylamine and N,N-diethylaminopropylamine, which are condensed with sodium chloroacetate. The use of a condensation product of C_{8/18} coconut fatty acid N,N-dimethylaminopropylamide with sodium chloroacetate is preferred.

Also considered as suitable starting materials for the betaines to be used in the context of the present disclosure are imidazolines which can be described by the formula

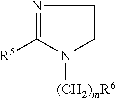

in which R⁵ stands for an alkyl radical having 5 to 21 carbon atoms, R⁶ stands for a hydroxyl group, an OCOR⁵ or NHCOR⁵ radical and m stands for about 2 or about 3. These substances are also known substances which can be obtained, for example, by cyclizing condensation of about 1 or about 2 moles of fatty acid with polyhydric amines, such as aminoethylethanolamine (AEEA) or diethylenetriamine. The corresponding carboxyalkylation products are mixtures of different open-chain betaines. Typical examples are condensation products of the abovementioned fatty acids with AEEA, preferably imidazolines based on lauric acid or again C_{12/14} coconut fatty acid, which are subsequently betainized with sodium chloroacetate.

Preferred cleaning agents as contemplated herein contain from about 0.5 to about 15% by weight, preferably from about 1 to about 10% by weight, more preferably from about 1.5 to about 7.5% by weight, even more preferably from about 2 to about 6% by weight and in particular from about 2.5 to about 5% by weight of surfactant(s) from the group of betaines.

The cleaning agents as contemplated herein can also contain aclyglutamate(s). These compounds are made from L-glutamic acid and naturally occurring fatty acids and are exemplified by low foaming and good detergency.

With particular preference, the cosmetic cleaning agents as contemplated herein contain from about 0.5 to about 15% by weight, preferably from about 1 to about 10% by weight, more preferably from about 1.5 to about 7.5% by weight, still preferably from about 2 to about 6% by weight and in particular from about 2.5 to about 5% by weight of acylglutamates of the formula (I)

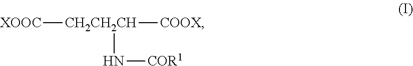

in which R¹CO stands for a linear or branched acyl radical having 6 to 22 carbon atoms and 0 and/or 1, 2 or 3 double bonds and X stands for hydrogen, an alkali and/or alkaline earth metal, ammonium, alkylammonium, alkanolammonium or glucammonium.

It is preferred in the context of the present disclosure to use alkali metal ions and in particular sodium ions as a cation X. In the case of fatty residues, the use of coconut fatty acids is particularly preferred and the use of raw materials made from palm oil should be dispensed with.

Very particularly preferred cosmetic cleaning agents as contemplated herein contain an acylglutamate in which X stands for Na and R¹CO stands for an acyl radical derived from coconut oil (INCI: Sodium Cocoyl Glutamate).

The cleaning agents as contemplated herein can also contain aclyglycinate(s). These compounds are made from L-glycine and naturally occurring fatty acids and are exemplified by very good foaming power and high skin friendliness.

With particular preference, the cosmetic cleaning agents as contemplated herein contain from about 0.5 to about 15% by weight, preferably from about 1 to about 10% by weight, more preferably from about 1.5 to about 7.5% by weight, still preferably from about 2 to about 6% by weight and in particular from about 2.5 to about 5% by weight of acylglycinates of the formula (II)

R¹CO—NH—CH₂—COOX (II),

in which R¹CO stands for a linear or branched acyl radical having 6 to 22 carbon atoms and 0 and/or about 1, about 2 or about 3 double bonds and X stands for hydrogen, an alkali and/or alkaline earth metal, ammonium, alkylammonium, alkanolammonium or glucammonium.

It is preferred in the context of the present disclosure to use alkali metal ions and in particular sodium ions as a cation X. In the case of fatty residues, the use of coconut fatty acids is particularly preferred and the use of raw materials made from palm oil should be dispensed with.

Very particularly preferred cosmetic cleaning agents as contemplated herein contain an acylglycinate in which X stands for Na and R¹CO stands for an acyl radical derived from coconut oil (INCI: Sodium Cocoyl Glycinate).

The agents as contemplated herein contain from about 0.1 to about 5% by weight of guaran (INCI name Guar Gum) as a second essential ingredient.

Guaran, also called guar gum, is a vegetable gum. The chemical compound from the group of polysaccharides is the main constituent of guar gum (or for short, guar flour). Guaran includes D-mannopyranose units, which are linked to each other in a chain via β-glycosidic bonds. In addition, every second mannopyranose unit carries α-D-galactopyranosyl radicals via a-bond.

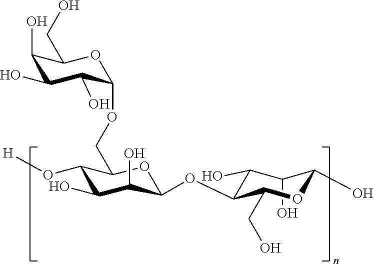

In combination with xanthan and modified and/or unmodified starches, guaran contributes to outstanding product properties and promotes applicability and product haptics.

Preferably, the guar gum is used in narrower ranges of amounts. Here, preferred cleaning agents as contemplated herein contain from about 0.2 to about 4% by weight, preferably from about 0.3 to about 3.5% by weight, more preferably from about 0.4 to about 3% by weight, even more preferably from about 0.45 to about 2.5% by weight and in particular from about 0.5 to about 2% by weight of guaran.

The agents as contemplated herein contain from about 0.1 to about 5% by weight of xanthan (INCI name Xanthan Gum) as a third essential ingredient. Xanthan gum is a natural, renewable resource and is excreted as an anionic polysaccharide by the bacterium Xanthomonas campestris.

The molecular weight of the xanthan gum used is preferably from about 2·10⁶ to about 20·10⁶ g/mol.

Xanthan gum contains D-glucose, D-mannose, D-glucuronic acid, acetate and pyruvate in an optional molar ratio of from about 28 to about 30 to about 20 to about 17 to about 5.1 to about 6.3 as molecular building blocks. The polymer backbone of xanthan gum is formed from a cellulose chain of β-1,4-linked glucose units. Xanthan contains structural units of the following formula

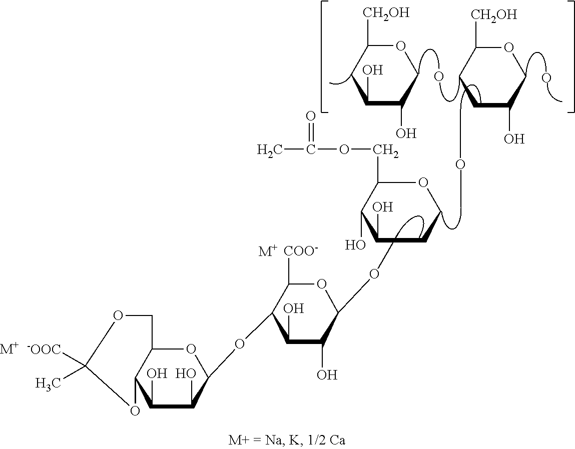

Heat-treated xanthan gum can also optionally be used in the cosmetic agents as contemplated herein.

In a preferred embodiment, the agent as contemplated herein contains as xanthan gum

heat-treated xanthan gum or

a mixture of xanthan gum and heat-treated xanthan gum.

When using a mixture of xanthan gum and heat-treated xanthan gum, it has been found to be particularly effective to use the xanthan gum and the heat-treated xanthan gum in a weight ratio range of from about 2 to about 1 to about 1 to about 20, in particular from about 1 to about 2 to about 1 to about 10.

As contemplated herein, heat-treated xanthan gum is understood as meaning xanthan gum which is exposed to heat of at least about 40° C. The resulting heat-treated xanthan gum has improved dispersibility and is more rapidly dispersed in water than xanthan gum which has not been subjected to heat treatment. The preferred suitable heat-treated xanthan gum has a viscosity of at least from about 25,000 to about 40,000 mPa·s (Brookfield DV-I viscometer, spindle #6 at about 23° C. and about 10 rpm) in a about 1% by weight aqueous solution. Preferably usable, heat-treated xanthan gums provide a pH value of from about 4.0 to about 6.0 at about 23° C. when preparing a about 1% by weight aqueous solution.

The preferred heat-treated xanthan gum as contemplated herein was obtained by tempering xanthan gum at a temperature of at least about 60° C., in particular of at least about 100° C. The tempering can be effected by a variety of known methods, such as by oven, fluidized bed, infrared or microwave heat treatment. In the context of the abovementioned heat treatments, it is again preferred when the xanthan gum has a water content of less than about 25% by weight, in particular less than about 8% by weight, very particularly preferably less than about 3% by weight before the heat treatment. It is further preferred as contemplated herein in the agent as contemplated herein to use such heat-treated xanthan gum, which was obtained in which xanthan gum having a water content of less than about 25% by weight at a temperature of at least about 60° C. (in particular of at least about 100° C.) is heat treated for at least about 30 minutes. It is particularly preferred as contemplated herein in the agent as contemplated herein to use such heat-treated xanthan gum, in which xanthan gum having a water content of less than about 8% by weight at a temperature of at least about 60° C. (in particular of at least about 100° C.) is heat treated for at least about 30 minutes. The preferred duration of the aforementioned heat treatments of xanthan gum, in particular with said water content, at a temperature of at least about 60° C. (in particular of at least about 100° C.) is at least about 1 hour. The particularly preferred duration of the abovementioned heat treatments of the xanthan gum, in particular having the preferred water content, at a temperature of at least about 60° C. (in particular of at least about 100° C.) is at least about 2.5 hours.

Preferably, the xanthan gum, whether it is heat-treated or not heat-treated or a mixture of both, is used in narrower ranges of amounts. Here, preferred cleaning agents as contemplated herein contain from about 0.2 to about 4% by weight, preferably from about 0.3 to about 3.5% by weight, more preferably from about 0.4 to about 3% by weight, even more preferably from about 0.45 to about 2.5% by weight and in particular from about 0.5 to about 2% by weight of xanthan.

The agents as contemplated herein contain from about 0.1 to about 10% by weight of modified and/or unmodified starch(es) as a further essential ingredient. In this case, the agents of the present disclosure

contain from about 0.1 to about 10% by weight of modified starch(es) and be free of unmodified starches;

contain from about 0.1 to about 10% by weight of unmodified starch(es) and be free of modified starches;

contain from about 0.1 to about 10% by weight of a mixture of modified and unmodified starch(es).

In other words, the agents as contemplated herein can contain both the ingredient d) and the ingredient e) or only one of the two ingredients. The total amount of starch(es) is as contemplated herein from about 0.1 to about 10% by weight in this case.

Starch (lat. Amylum) is an organic compound. It is a polysaccharide of the formula (C₆H₁₀O₅)ₙ including α-D-glucose units.

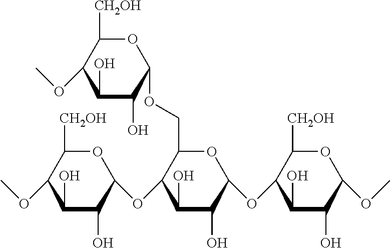

Modified starches are starch products obtained by physical, enzymatic or chemical processes that meet increased technical requirements. The grain structure and other essential properties are retained after the modification.

The raw material for the production of modified starch is natural or degraded starch, which is converted by polymer-analogous reactions into the respective derivatives. The natural starch is subjected to various chemical transformation processes, depending on which properties are to be changed. For some modified starches, several transformation processes are performed one after the other:

acid-treated starch by reaction with acids

alkali-modified starch by reaction with alkalis

bleached starch by treatment with peroxyacetic acid, hydrogen peroxide, sodium

hypochlorite, sodium chlorite, sulfur dioxide, sulphites, potassium permanganate or ammonium persulphate

enzymatically modified starch by treatment with amylases

oxidized starch by oxidation (for example, with sodium hypochlorite)

monostarch phosphate by esterification with phosphorous ester groups (for example, phosphoric acid, sodium or potassium phosphate, phosphonic acid or pentasodium triphosphate)

distarch phosphate by esterification with sodium trimetaphosphate or phosphorus oxychloride

phosphated distarch phosphate by combining the methods for producing monostarch phosphate and distarch phosphate

starch acetate or acetylated starch by reaction with acetic acid anhydride or esterification with acetic acid

hydroxypropyl starch by reaction with propylene oxide

starch sodium octenylsuccinate by reaction of starch with octenyl succinic anhydride Improvements in resistance to heat, cold and/or pH changes (acids) are most frequently sought.

It is particularly preferred as contemplated herein to use modified starch(es) and to keep the content of unmodified starch rather low. In general, agents are preferred which have a higher weight fraction of modified starch with respect to the weight fraction of unmodified starch. Particularly preferred is the weight ratio of unmodified to modified starch(es) greater than about 1, preferably greater than about 5, more preferably greater than about 10 and in particular greater than about 25.

Particularly preferred cosmetic cleaning agents as contemplated herein contain

from 0 to about 5% by weight, preferably from 0 to about 2.5% by weight, more preferably from 0 to about 1% by weight, even more preferably from 0 to about 0.5% by weight and in particular less than about 0.1% by weight of unmodified starch and

from about 0.1 to about 10% by weight, preferably from about 0.25 to about 9% by weight, more preferably from about 0.5 to about 8% by weight, still more preferably from about 0.75 to about 7% by weight and in particular from about 1 to about 6% by weight of modified starch(es).

Very particular preference is given to the use of hydroxypropyl starch and/or hydroxypropyldistarch phosphate. Extremely preferred cosmetic cleaning agents as contemplated herein therefore contain, based on their weight, from about 0.1 to about 10% by weight, preferably from about 0.25 to about 9% by weight, more preferably from about 0.5 to about 8% by weight, even more preferably from about 0.75 to about 7% by weight and in particular from about 1 to about 6% by weight of hydroxypropyl starch and/or hydroxypropyldistarch phosphate.

The object of the present disclosure is excellently achieved by the combination of the four or five ingredients. The use of synthetic thickeners is therefore no longer necessary and in view of the task, in particular the consumer needs, rather counterproductive. The present disclosure provides products with the power and haptics known to the consumer, without having to resort to synthetic thickeners.

It is therefore preferable to keep the agents as contemplated herein largely free of the substances mentioned. Cosmetic cleaning agents preferred as contemplated herein do not contain acrylate polymers and do not contain acrylate copolymers.

Even more preferred cosmetic cleaning agents as contemplated herein do not contain fully synthetic polymers.

A further object of the present disclosure is the use of mixtures of

guaran (INCI name Guar Gum);

xanthan (INCI name Xanthan Gum);

modified and/or unmodified starch

for thickening cosmetic cleaning agents.

With respect to preferred embodiments of the use as contemplated herein mutatis mutandis applies to the agent of the present disclosure.

### EXAMPLES

The following composition can be produced by way of example:

| | 1 | 2 | 3 |
|---|---|---|---|
| Cocamidopropyl betaine | 4.0 | 4.0 | 4.0 |
| Coco glucoside | 1.4 | 1.3 | 1.4 |
| Caprylyl/caprylic glucoside | 0.4 | 0.4 | 0.4 |
| Sodium Laureth Sulfate | 4.0 | — | — |
| Disodium Laureth Sulfosuccinate | — | 4.0 | — |
| Sodium Methyl Cocoyl Taurate | — | — | 4.0 |
| Cyamopsis Tetragonoloba (Guar) Gum | 0.5 | 0.45 | 0.5 |
| Xanthan Gum | 0.5 | 0.6 | 0.5 |
| Hydroxypropylated distarch phosphate | 4.0 | 3.8 | 4.0 |
| Glycerin | 5.0 | 5.0 | 5.0 |
| Sorbitol | 2.0 | 2.0 | 2.0 |
| Kaolin | 0.7 | 0.7 | 0.7 |
| Citric acid | 0.5 | 0.5 | 0.5 |
| Sodium chloride | 0.5 | 0.5 | 0.5 |
| Ricinus Communis (Castor) seed oil | 0.5 | 0.5 | 0.5 |
| Helianthus Annuus (Sunflower) seed oil | 0.4 | 0.4 | 0.4 |
| Sodium benzoate | 0.3 | 0.3 | 0.3 |
| Sodium salicylate | 0.2 | 0.2 | 0.2 |
| Butyrospermum Parkii (Shea) Butter | 0.2 | — | 0.33 |
| Guar hydroxypropyltrimonium chloride | 0.2 | — | 0.2 |
| Sodium hydroxide | 0.15 | — | 0.15 |
| CI 77891 (titanium dioxide) | 0.1 | — | 0.09 |
| Allantoin | 0.03 | — | — |
| Chamomilla Recutita (Matricaria) flower | 0.02 | — | — |
| extract | | | |
| Water | ad 100% | ad 100% | ad 100% |
| | 4 | 5 | 6 |
| Coco betaine | 4.5 | 4.0 | 3.8 |
| Coco glucoside | 1.4 | 1.3 | 1.4 |
| Sodium Methyl Cocoyl Taurate | 3.5 | 4.0 | |
| Sodium Lauroyl Methyl Isethionate | — | — | 4.3 |
| Cyamopsis Tetragonoloba (Guar) Gum | 0.5 | 0.45 | 0.5 |
| Xanthan Gum | 0.5 | 0.6 | 0.5 |
| Hydroxypropylated distarch phosphate | 5.0 | 4.8 | 4.0 |
| Glycerin | 5.0 | 5.0 | 5.0 |
| Sorbitol | 2.0 | 2.0 | 2.0 |
| Citric acid | 0.5 | 0.5 | 0.5 |
| Sodium chloride | 0.5 | 0.5 | 0.5 |
| Sodium benzoate | 0.3 | 0.3 | 0.3 |
| Sodium salicylate | 0.2 | 0.2 | 0.2 |
| Butyrospermum Parkii (Shea) Butter | 0.2 | — | 0.33 |
| Guar hydroxypropyltrimonium chloride | 0.2 | — | 0.2 |
| Sodium hydroxide | 0.15 | — | 0.15 |
| CI 77891 (titanium dioxide) | 0.1 | — | 0.09 |
| CI 47005 (Yellow 10) | — | 0.1 | — |
| Allantoin | 0.03 | — | — |
| Chamomilla Recutita (Matricaria) flower | 0.02 | — | — |
| extract | | | |
| Water | ad 100% | ad 100% | ad 100% |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic cleaning agent comprising, based on a total weight of the cosmetic cleaning agent,
a) from about 1 to about 30% by weight of surfactant;
b) from about 0.1 to about 5% by weight of guaran;
c) from about 0.1 to about 5% by weight of xanthan;
d) from 0 to about 10% by weight of unmodified starch; and
e) from 0 to about 10% by weight of modified starch,
wherein a total amount of ingredients d) and e) is from about 0.1 to about 10% by weight.

2. The cosmetic cleaning agent according to claim 1, wherein the cosmetic cleaning agent comprises from about 1.5 to about 25% by weight of the surfactant.

3. The cosmetic cleaning agent according to claim 1, wherein the surfactant is selected from the group of alkyl oligo- and polysaccharides,
sulfosuccinates,
isethionates,
taurides,
aclyglutamate,
glycinates,
betaines, or combinations thereof.

4. The cosmetic cleaning agent according to claim 1, wherein the cosmetic cleaning agent comprises from about 0.2 to about 4% by weight of the guaran.

5. The cosmetic cleaning agent according to claim 1, wherein the cosmetic cleaning agent comprises from about 0.2 to about 4% by weight of the xanthan.

6. The cosmetic cleaning agent according to claim 1, wherein the cosmetic cleaning agent comprises,
from about 0 to about 5% by weight of the unmodified starch and
from about 0.1 to about 10% by weight of the modified starch.

7. The cosmetic cleaning agent according to claim 1, wherein the cosmetic cleaning agent comprises from about 0.1 to about 10% by weight of the modified starch, and wherein the modified starch is selected from the group of hydroxypropyl starch, hydroxypropyl distarch phosphate, or combinations thereof.

8. The cosmetic cleaning agent according to claim 1, wherein the cosmetic cleaning agent does not comprise acrylate polymers, and wherein the cosmetic cleaning agent does not comprise acrylate copolymers.

9. The cosmetic cleaning agent according to claim 1, wherein the cosmetic cleaning agent does not contain fully synthetic polymers.

10. The cosmetic cleaning agent of claim 2, wherein the cosmetic cleaning agent comprises from about 2 to about 20% by weight of the surfactant.

11. The cosmetic cleaning agent of claim 10, wherein the cosmetic cleaning agent comprises from about 2.5 to about 15% by weight of the surfactant.

12. The cosmetic cleaning agent of claim 4, wherein the cosmetic cleaning agent comprises from about 0.3 to about 3.5% by weight of the guaran.

13. The cosmetic cleaning agent of claim 12, wherein the cosmetic cleaning agent comprises from about 0.4 to about 3% by weight of the guaran.

14. The cosmetic cleaning agent of claim 5, wherein the cosmetic cleaning agent comprises from about 0.3 to about 3.5% by weight of the xanthan.

15. The cosmetic cleaning agent of claim 14, wherein the cosmetic cleaning agent comprises from about 0.4 to about 3% by weight of the xanthan.

16. The cosmetic cleaning agent of claim 6, wherein the cosmetic cleaning agent comprises less than about 0.1% by weight of the unmodified starch.

17. The cosmetic cleaning agent of claim 6, wherein the cosmetic cleaning agent comprises from about 0.25 to about 9% by weight of the modified starch.

18. The cosmetic cleaning agent of claim 17, wherein the cosmetic cleaning agent comprises from about 0.5 to about 8% by weight of the modified starch.

19. The cosmetic cleaning agent of claim 18, wherein the cosmetic cleaning agent comprises from about 0.75 to about 7% by weight of the modified starch.

20. The cosmetic cleaning agent of claim 7, wherein the cosmetic cleaning agent comprises from about 1 to about 6% by weight of the modified starch.
